(19) United States
(12) Patent Application Publication
(10) Pub. No.: US 2009/0142282 A1
(43) Pub. Date: Jun. 4, 2009

(54) High-solids alcoholic solutions of rosin esters for varnish applications

(76) Inventors: Jonathan Lee Kendall, Cary, NC (US); Donald Wilson Kendall, Charlotte, NC (US)

Correspondence Address: Jonathan Kendall, 616 Halcyon Meadow Dr., Cary, NC, 27519, US

(21) Appl. No.: 12/315,208

(22) Filed: Dec. 1, 2008

Related U.S. Application Data
(60) Provisional application No. 61/005,215, filed on Dec. 4, 2007.

(51) Int. Cl. A61K 8/97; A61K 8/21; A61Q 11/00; A61K 8/33

(52) U.S. Cl. 424/52; 424/49; 424/195.18

(57) ABSTRACT

High-solids solutions of solid rosin esters and lower alcohols can be obtained at rosin ester concentrations above the binary solubility of the rosin ester in the alcohol. Such solutions can be achieved by adding an additional modified rosin or by dissolving a small amount of the alcohol in the rosin ester. These solutions are useful as varnishes for use on humans and animals, particularly when ethanol is the alcohol. The varnish may optionally contain a therapeutic agent.

### CROSS-REFERENCE TO RELATED APPLICATIONS

This application claims the benefit of provisional patent application Serial Number 61/005,215, filed Dec. 4, 2007 by the present inventors.

### FEDERALLY SPONSORED RESEARCH

Not Applicable

### SEQUENCE LISTING OR PROGRAM

Not Applicable

### BACKGROUND

1. Field of Invention

This invention relates to obtaining high-solids solutions of rosin esters (especially hydrogenated rosin esters) in C₂-C₄ alcohols, preferably in ethanol. Specifically, it relates to the use of such solutions in varnishes for protective and/or therapeutic use on humans or animals.

2. Prior Art

Varnishes based on natural resins have been used since ancient times to produce solid transparent or semi-transparent coatings with protective and/or decorative properties. Among the natural resins, rosin has found widespread use in varnishes because of the superior properties, such as water repellency, abrasion resistance, adhesiveness, film formation, and integrity, which it imparts to coatings.

Rosin and its derivatives are particularly attractive for use in varnishes applied to humans and animals. They have a long history as additives in chewing gum and personal care products. They are used as direct additives in foods and inactive ingredients in topical and oral pharmaceuticals. Rosin-based varnishes are useful when a removable, water-insoluble coating with good adhesion to human or animal tissue is required. They are particularly attractive when the varnish may be ingested or otherwise enter the body.

Because of their ease of application and excellent coating properties, varnishes are useful in dental applications. A dental varnish may act as a barrier alone or it may additionally contain a therapeutic agent. Dental varnishes are usually spirit varnishes wherein a film-forming, water-insoluble resin or polymer (natural or synthetic) is dissolved in a pharmaceutically acceptable solvent and applied to the desired tooth area. Varnishes that are most desirable do not require thorough drying of the teeth before application. The solvent evaporates and/or is extracted by saliva to leave a coating that remains on the tooth surface for at least several hours.

Commercial dental varnishes are well known in the art. The first dental varnishes, used to coat prepared cavity walls prior to placement of restorative materials, were based on natural copal resins dissolved in chlorinated hydrocarbons and are still used to prevent penetration of restorative materials into dental tissue.

Particularly useful dental varnishes contain fluoride, which are usually administered to patients who are at high risk for dental carries. Fluoride varnishes are approved by the FDA as medical devices for use as cavity preparation liners and for the treatment of hypersensitive teeth.

Despite the numerous advantages of dental varnishes, they still have limited use on dental patients compared to fluoride gels, foams, and rinses. An important disadvantage of traditional sodium fluoride varnishes based on natural resins is that the yellow to brown color of the natural resin causes a temporary change in tooth color. This undesirable characteristic limits patient acceptance of these natural resin varnishes and has promoted efforts to make varnishes based on modified natural resins in more acceptable lighter colors to increase patient acceptance.

Modified rosins that have been esterified and/or hydrogenated are available commercially in exceptionally light colors. These modified rosins can produce a colorless or near-colorless varnish. Unfortunately, compared to natural rosin, these materials have significantly lower solubility in solvents that are pharmaceutically acceptable for oral use. Ideally, solvents used in preparations that enter the body have acceptable taste, irritation, toxicity, and allergic reaction (sensitization) profiles. The most commonly used solvents in oral preparations requiring water miscibility are ethanol, glycerin, propylene glycol, and PEG 300 & 400. Applications for water immiscible solvents most often use liquid paraffin and vegetable oils. For topical use, saturated aliphatic hydrocarbons, ether, and isopropanol, among others, are also acceptable solvents.

Although a dental varnish is topical in its application, its prolonged residence in the oral cavity results in unavoidable and unintended ingestion, particularly the portion of the solvent that does not evaporate during the initial application. The ideal solvent would be low in toxicity, volatile enough to allow rapid drying of the varnish, water soluble, and extractable by bodily fluids, such as saliva Since ethanol possesses these properties, it is a preferred solvent for applications where the varnish may enter the body, such as dental varnishes.

### SUMMARY OF THE INVENTION

In a first aspect, the present invention is a varnish composition comprising a solution of a solid rosin ester and at least one natural or modified rosin in a liquid, which is volatile, water soluble, or both, comprising at least one C₂₋₄ alcohol, wherein the amount of solid rosin ester and the natural or modified rosin present in the composition is greater than the solubility of the solid rosin ester alone in the liquid.

In a second aspect, the present invention is a varnish composition comprising a solution of a solid rosin ester and a liquid, which is volatile, water soluble, or both, comprising at least one C₂₋₄ alcohol, wherein the amount of solid rosin ester present in the composition is greater than the solubility of the solid rosin ester in the liquid.

In a third aspect, the present invention is a method of applying a varnish composition of the first and second aspects by use of a brush, cloth, gauze, swab, syringe, flexible support (for example, a tape, patch, or matrix), dental tray, or other suitable applicator.

The ethanol solubility of rosin esters, especially the desirable highly hydrogenated rosin esters, can be improved significantly by combining them with certain other modified rosins. Additionally, there are one-phase liquids that are useful over a limited concentration range at very high rosin ester concentration. The present invention makes it possible to formulate high-solids rosin varnishes that have the desirable properties of low or no color with sufficient viscosity to suspend therapeutic agents in a water-miscible, pharmaceutically acceptable solvent for oral applications. Such compositions also avoid long drying times, minimize solvent ingestion, and promote good film-forming properties, for example, anti-sagging.

### DETAILED DESCRIPTION OF THE INVENTION

### Definitions

Varnish means a liquid preparation that when spread and allowed to dry on a surface forms a typically hard, typically transparent coating that exhibits protective, decorative, and/or therapeutic properties. Though varnish traditionally implies a transparent or semi-transparent coating upon drying, as used herein, it also includes opaque films.

Resin is a general term denoting a wide variety of natural and synthetic products meaning any of various solid or semi-solid amorphous organic substances that are usually transparent or translucent, are formed especially in plant secretions, are usually soluble in organic solvents but not in water, and are used chiefly in varnishes, printing inks, plastics, sizes, and medicine.

Natural resin means resin produced by natural sources, consisting largely of resin acid, anhydride, and ester mixtures, and may also be mixtures with gums and essential oils. Examples include, but are not limited to, rosin, copal, dammar (damar), shellac (lac), mastic, sandarac, kauri, and amber.

Natural rosin means a translucent natural resin that is composed chiefly of rosin acids (such as abietic acid and its isomers and derivatives) and is obtained by distillation or extraction of the oleoresin [viscous liquid that exudes from the tree when wounded] of living (gum rosin) or dead (wood rosin) pine trees by removal of volatile oils or from tall oil (tall oil rosin) by removal of fatty acids. Natural rosin is sometimes referred to as gum rosin, wood rosin, tall oil rosin, colophony, and resin (though not properly and not herein).

Modified rosin(s) means the product(s) of natural rosin treated with heat, catalytically, stoichiometrically, or combinations thereof in the presence or absence of chemically reactive agents to change the structure of natural rosin. Examples include disproportionated (dehydrogenated) rosin, heat-treated rosin, hydrogenated rosin, dimerized rosin, polymerized rosin, esterified rosin (rosin ester), hydroabietyl alcohol, rosin salts, and combinations thereof. Herein, modified rosin(s) also means rosin derivative(s).

Hydrogenated rosin means rosin that has reacted with hydrogen to reduce ethylenic unsaturated bonds. Both partially hydrogenated and highly hydrogenated rosins are available commercially.

Rosin ester means rosin that has been modified by esterification reaction with alcohol(s). The rosin ester may contain a small amount of residual unreacted acid and/or unreacted alcohol groups. The alcohol(s) may be mono-, di-, tri-, or higher-hydridic. Exemplary alcohols include, but are not limited to, methanol, triethylene glycol, glycerol, and pentaerythritol.

Hydrogenated rosin ester means a modified rosin that satisfies the definitions above for both hydrogenated rosin and rosin ester.

High solids means a composition that, under normal atmospheric or biologic conditions, contains at least one non-volatile (and, where applicable, water insoluble) component and at least one volatile component whereby the non-volatile (and, where applicable, water insoluble) component(s) comprise at least 60% by weight of the total composition. In this context, the non-volatile component(s) may be a solid or a liquid provided that it remains in the film after the varnish has cured.

Volatile liquid means an ambient liquid with a vapor pressure of at least about 1 mm Hg at 25° C.

Water soluble liquid means an ambient liquid with a water solubility of at least about 3 g/100 mL of water at 25° C. and includes liquids miscible with water.

Stable solution means a one-phase liquid mixture, as observed by the human eye, which remains one phase under ambient conditions for at least three months, preferably at least six months, and most preferably at least one year.

C₂-C₄ alcohol means a compound containing 2-4 carbon atoms and at least one hydroxyl group. Exemplary C₂-C₄ alcohols include, but are not limited to, ethanol, 1-propanol, 2-propanol (isopropanol), propylene glycol, glycerin (glycerol), 1-butanol, 2-butanol, and 2-methyl-2-propanol (tert-butyl alcohol).

Pharmaceutically acceptable solvent means a solvent that, when used in amounts common for pharmaceutical preparations, does not produce a biological reaction reflected by infections, inflammations, or other phenomena of rejection.

Pharmaceutically acceptable solvent for oral use means a solvent that satisfies the definition for pharmaceutically acceptable solvent for uses that involve ingestion into the human (or animal) body.

### Rosin Solubility

Saturated solutions were prepared by warming the modified rosin and the alcohol or other liquid to prepare a saturated solution. In some cases, all of the rosin dissolved and no precipitate formed upon cooling, giving the impression that the solution was stable. However, after sitting for several days or weeks, a precipitate formed indicating that the solutions were only metastable and sufficient time was required to pass before the solubilities could be determined. This observation is significant because in order to have a commercially practical shelf life, solutions to be used in varnishes must be stable for at least three months under ambient conditions.

In contrast to natural rosin, the solubility of rosin esters, hydrogenated esters, and hydrogenated rosins are somewhat poor in ethanol. In our hands, natural rosin solutions in ethanol can be prepared at about 80-90% solids (w/w) [All solubility and percent solid values in this disclosure are given in w/w, unless otherwise indicated]. The solubility of commercially available partially hydrogenated rosin, glycerol ester of rosin, and glycerol ester of partially hydrogenated rosin in ethanol were found to be about 65-70, 40-45, and 30-35 wt. % solids, respectively. The solubility of the more desirable highly hydrogenated rosin and rosin glycerol esters are even lower (45-50% and 25-30%, respectively). Pentaerythritol esters of hydrogenated rosins have particularly low solubilities: 15-20% for partially hydrogenated and 10-20% for highly hydrogenated. The present invention allows for the formation of high solids solutions of rosin esters and hydrogenated rosin esters in C₂-C₄ alcohols, particularly ethanol.

One strategy involves mixing these modified rosins with liquid modified rosins. A stable solution of 10% hydroabietyl alcohol and 65% rosin glycerol ester was prepared in ethanol. Another liquid rosin derivative, hydrogenated rosin methyl ester, enabled the preparation of stable ethanolic solutions with several rosin esters. A 75% solids solution was prepared from rosin glycerol ester using 5% hydrogenated rosin methyl ester. An 80% solids solution was prepared from 75% hydrogenated rosin glycerol ester and 5% hydrogenated rosin methyl ester. Even the less soluble hydrogenated rosin pentaerythritol ester formed a 70% solids solution with the hydrogenated rosin methyl ester, though the latter made up one-fourth of the total solids.

High solids solutions in ethanol can also be made from solid rosin esters and solid hydrogenated rosin. A number of stable solutions of up to 84% solids were formed with non-, partially, and highly hydrogenated glycerol esters of rosin mixed with hydrogenated rosin over a range of concentrations. An essentially colorless solution was prepared from 25% highly hydrogenated rosin and 50% highly hydrogenated rosin glycerol ester. A particularly high solids solution was prepared from 16% ethanol, 28% hydrogenated rosin, and 56% highly hydrogenated rosin glycerol ester. This solution was very light in color, and no precipitate had formed after two years storage under ambient conditions.

Interestingly, even though the rosin solubility studies indicated that the acid forms of rosin were much more soluble than the rosin esters, the solubility of mixtures of these components were somewhat more sensitive to the concentration of the rosin acid. However, the amount of rosin acid that could be present and still form a stable solution depended on the nature of the rosin acid and the rosin ester.

Stable solutions were also obtained with hydrogenated pentaeryritol esters and hydrogenated rosins. In some cases, two phases formed immediately after cooling, but became one phase over the course of one month and stayed one phase for at least three months. The initial phase separation problem could be avoided by agitating the solution during cooling.

Alcohols and alcohol mixtures other than pure ethanol can also be used to form high solids solutions. Highly hydrogenated rosin and its glycerol ester are soluble in isopropanol at about 60-65 and 80-85%, respectively. A very viscous solution was prepared from these modified rosins at 88% solids in a 1:3 rosin acid/rosin ester ratio in isopropanol. These rosins also formed solutions at 75% solids in an ethanol/isopropanol mixed solvent system and at 66% in an ethanol/glycerol mixed solvent system.

Another strategy for making high solids solutions was found during the course of the solubility determination experiments. It was observed that the rosin esters, in contrast to the rosin acids, formed a viscous lower liquid layer instead of a precipitate when the rosin ester was present above the solubility of the rosin ester in ethanol. This implied that an ethanol-rich liquid and a rosin ester-rich liquid were being formed. Solubility investigations at much higher rosin ester concentrations revealed a one-phase composition at rosin ester concentrations well above the solubility found for the rosin ester in ethanol. These one-phase compositions occurred at glycerol ester of partially hydrogenated rosin amounts above 70-75% and glycerol ester of highly hydrogenated rosin amounts above 75-80%. Viscous stable solutions of partially hydrogenated rosin glycerol ester and ethanol were prepared at 75% and 80%. A composition containing 88% partially hydrogenated rosin glycerol ester and 12% ethanol was still a liquid, though an extremely viscous one, but the composition containing 98% of this glycerol ester and 2% ethanol was a solid at 25° C.

### Viscosity

In some applications it is desirable for the varnish to have sufficient viscosity to prevent excessive spreading or sagging on the substrate receiving the varnish and/or to minimize settling of insoluble additives, such as sodium fluoride, during storage to give the varnish a commercially useful shelf life. Though viscosity can be increased by the addition of thickeners such as fumed silica or polymeric materials, it is desirable to have a rosin solution of a certain minimum viscosity to minimize any adverse adhesion or film integrity effects high levels of additives may have on the varnish. The viscosity of a number of rosin solutions were measured via the falling ball method and compared to the benchmark composition 75% natural rosin/25% ethanol, which was selected as a control because a number of commercial dental varnishes have a similar base formulation.

The viscosity of the varnish comprising 50% partially hydrogenated rosin/25% partially hydrogenated rosin glycerol ester was about 15% higher than the control. Though both formulations contain 75% solids, the presence of the higher molecular weight glycerol ester should impart a higher viscosity than the natural rosin. This point is further exemplified by the five-fold higher viscosity of 75% partially hydrogenated rosin glycerol ester. The addition of 2% fumed silica to the rosin acid/rosin ester formulation above increased its viscosity by a factor of about six. The higher solids formulations 75% hydrogenated rosin glycerol ester/5% hydrogenated rosin methyl ester and 28% hydrogenated rosin/56% hydrogenated rosin glycerol ester had viscosities over an order of magritude higher than the control. Thus, the solubilization strategies presented here can result in solutions of sufficient viscosity to prevent excessive spreading/sagging and for applications requiring the suspension of insoluble materials.

### Coating Adhesion

In order for the rosin solutions to be useful in varnishes, they must be able to produce good films that are stable in their use environment. For example, in dental applications, the varnish must adhere to teeth for hours in an aqueous environment at about 37° C. To test film formation and adhesion, rosin solutions were applied to extracted human teeth with nylon brushes. The films were allowed to dry for a few minutes and then were placed in a 37° C. stirred water bath for six hours. The films were removed and inspected visually and with a wooden probe.

The control varnish of 75% natural rosin/25% ethanol formed a smooth coating that stayed attached to the teeth during the stirred water experiment and required some effort to be removed with the wooden probe. Thicker films were softer than thinner films, which reflected incomplete solvent evaporation and/or extraction. Results similar to that of the control were observed for ethanol solutions of partially hydrogenated rosin/partially hydrogenated rosin glycerol ester (with and without fumed silica), highly hydrogenated rosin/highly hydrogenated rosin glycerol ester, highly hydrogenated rosin/highly hydrogenated rosin pentaerythritol ester, partially hydrogenated rosin methyl ester/partially hydrogenated rosin glycerol ester, and rosin glycerol ester/hydroabeityl alcohol. The solution of 25% ethanol in partially hydrogenated rosin glycerol ester gave similar results. Thus, the solubilization strategies presented here can result in solutions of sufficient adhesion to teeth in an aqueous environment at human body temperature, as would be required for a dental varnish.

For comparison, two additional natural resin compositions were prepared and tested. A 75% partially hydrogenated rosin glycerol ester in a 70/30 (v/v) hexanes/ethanol solution had similar adhesion as the natural rosin control. However, thicker films exhibited air pockets and had a bumpy appearance. This was probably caused by water-insoluble hexane that had not evaporated during the three-minute drying period before being placed in the water. Another comparative composition tested was 65% refined bleached shellac in ethanol. This varnish produced a textured, white, opaque coating that was very difficult to remove from the teeth using the wooden probe. Such strong adhesion may cause problems in cases where it is desirable for the patient to remove the coating with simple teeth brushing. Additionally, the bright white appearance of the opaque coating is undesirable because it is strikingly different from the appearance of natural teeth.

Adhesion experiments were performed to determine the suitability of rosin ester varnishes on soft tissue. The 25% ethanol in partially hydrogenated rosin glycerol ester varnish was found to adhere to human gingiva in vivo. Gentle rubbing did not remove the coating. After 2 h it was easily be removed by hand. Similarly, this varnish adhered to human skin, surviving multiple hand washings. The film was peeled away by hand after 6 h.

### Varnish Composition

The varnish of this invention may contain additional components. The resin mixture may include other natural or synthetic resins, including, but not limited to, cumarone, copal, dammar (dammar), shellac (lac), mastic, sandarac, kauri, amber, polyurethane, polyamide, and ethyl cellulose. Such other resins are preferably present at about 1-50%.

Ethanol is the preferred alcohol, but additional alcohols may be used alone or in conjunction with ethanol. Additional preferred alcohols are those that are pharmaceutically acceptable for oral use such as glycerin (glycerol) and propylene glycol. Other acceptable alcohols are the C₂-C₄ alcohols. Examples include 1-propanol, 2-propanol (isopropyl alcohol), 1-butanol, 2-butanol (sec-butyl alcohol), and 2-methyl-2-propanol (tert-butyl alcohol). Higher alcohols have substantially longer evaporation times, which cause increased drying time. In some cases, the higher alcohols additionally have limited water solubility, which would inhibit their extraction by saliva. The lower alcohol methanol is not preferred because of its adverse human effects. The preferred alcohol is ethanol because it is long history as a preferred solvent in oral pharmaceutical applications, its water solubility, and its optimal vapor pressure that allows efficient drying of the varnish without being too volatile to limit shelf life or require excessive packaging to obtain an acceptable shelf life.

The varnish may contain other liquids in addition to C₂-C₄ alcohols as needed to optimize performance such as drying time or coating formation. Such liquids are preferably those that meet the definition of pharmaceutically acceptable solvents and the desirable vapor pressure characteristics mentioned above. For certain oral applications, extraction by saliva may lessen the volatility requirements if the additional liquid is water soluble, particularly if it is present in a small amount (<5%).

The volatile components in the varnish may be present in at least 10% by weight, including, about 15%, about 20% about 25%, about 30%, about 35%, and about 40%, and also including percentages between those recited.

The varnish formulations optionally comprise a fluoridizing agent. Fluoridizing agents include sodium fluoride, stannous fluoride, sodium monofluoro phosphate, zinc hexafluorosilicate, ammonium fluorides, and sodium hexafluorosilicate. When present, there is preferably about 0.1% to 10% fluoridizing agent by weight.

The varnish formulations optionally comprise other therapeutic and/or cosmetic agents such as tooth whitening agents like urea hydrogen peroxide, carbamide peroxide, and peroxyborate monohydrate; anti-hypersensitivity agents like potassium nitrate, sodium nitrate, strontium salts, potassium bicarbonate, hydroxyapatite, and fluoroapatite; and anti-microbial agents such as chlorhexidine, cetylpyridinium chloride, and benzalkonium chloride.

The varnish formulations optionally comprise one or more of the following: sweeteners such as xylitol, sorbitol, aspartame, sodium saccharin, and mixtures thereof; flavorings including, but not limited to, peppermint oil, cherry, citric acid, orange, strawberry, vanilla, coconut, bubble gum flavors, spearmint oil, raspberry, and mixtures thereof; and coloring agents. When a sweetener is present, it is preferably present at 0.1-4% by weight or some other quantity sufficient to impart an improved palatability to the varnish. When flavorings are present they are preferably present at 0.1-4% by weight.

The varnish may optionally contain fillers and/or thickening agents. Fillers and/or thickeners may be organic or inorganic and may be polymeric or non-polymeric. Exemplary fillers and/or thickeners include, but are not limited to, silica, fumed silica, alumina, polyethylene glycol, polypropylene glycol, glycerin, titanium oxide, and zinc oxide. When a filler or thickener is present, it is preferably present at 0.5-15% by weight.

### Varnish Application and Storage

The varnishes described may be applied using a suitable applicator, as is well understood by those skilled in the art. For dental applications, there are known methods to apply a varnish to a tooth and allowing the solvent to evaporate to leave behind a film that covers and/or seals all or part of the tooth.

Application methods include, but are not limited to, painting with a brush, cloth, gauze, or swab, delivery through a syringe, and spreading with a wooden, metal, or plastic applicator. The varnish may additionally be applied using a dental tray, wherein the varnish is pre-applied to the surfaces of the tray prior to delivery to the dentition. The varnish may also be applied to a flexible support (porous or solid; tape or patch or matrix). The varnish-coated flexible support can then be delivered to the desired surface(s). The flexible support may be removed or left on or in the varnish.

The application method may depend on the circumstances of the varnish use. When small, difficult to reach surfaces are receiving the varnish, applicators with small tips are needed. When speed of delivery is the most pressing need, for example, in dental applications requiring a dry tooth field, the syringe, flexible support, or tray delivery methods may be appropriate.

The varnishes described may be contained for shipment, storage, and use in a number of package types. The container may be a can, jar, pouch, syringe, tube, or other convenient and/or appropriate vehicle.

The invention is further described in the examples, which are given solely for the purpose of illustration, and are not intended to limit its scope as defined in the patent claims.

### EXAMPLES 1-8

### Solubility Determination

The solubilities of commercial natural rosin, rosin esters, hydrogenated rosin esters, and hydrogenated rosins were determined by warming a 50-85% mixture in a solvent to boiling or near boiling. In some cases, the rosin completely dissolved and initially stayed in solution upon cooling, but precipitated over the course of several days or weeks. Thus, solubility determination had to be delayed to ensure that solutions were saturated and not supersaturated. Triplicate aliquots of saturated solutions were oven dried to a constant weight to determine the rosin solubility.

| | | | Solubility at room temperature | | | |
|---|---|---|---|---|---|---|
| | | | | | | Hexane/ |
| Example | Rosin Type | Hydrogenation | Ethanol | IPA | Hexane | Ethanol^{†} |
| 1 | natural rosin | none | 80-90% | | | |
| 2 | rosin acid | partial | 65-70% | 70-75% | | 55-60% |
| 3 | rosin acid | high | 45-50% | 60-65% | | 45-55% |
| 4 | glycerol ester | none | 40-45% | | | |
| 5 | glycerol ester | partial | 30-35% | 80-85% | 85-90% | 85-90% |
| 6 | glycerol ester | high | 25-30% | 80-85% | 85-90% | 85-90% |
| 7 | pentaerythritol ester | partial | 15-20% | | | |
| 8 | pentaerythritol ester | high | 10-20% | | | |

^{†} Hexane/ethanol in 70%/30% (v/v).

### Solution Studies

For the solution studies of Examples 9-34, samples were prepared by combining all contents in a vial and heating to near boiling until a one-phase liquid had formed. Samples were cooled while being agitated on a mechanical roller or by hand. Solution stability was determined visually over three-month storage at room temperature.

### EXAMPLES 9-15

### Solid Rosin/Liquid Rosin Solutions in Ethanol

| | Solid Rosin | | | Liquid Rosin | | Stable |
|---|---|---|---|---|---|---|
| Example | Amount | Rosin Type | Hydrogenation | Amount | Rosin Type* | solution? |
| 9 | 65% | acid | partial | 15% | alcohol | no |
| 10 | 65% | glycerol ester | none | 10% | alcohol | yes |
| 11 | 67% | acid | partial | 9% | methyl ester | no |
| 12 | 70% | glycerol ester | none | 5% | methyl ester | yes |
| 13 | 75% | glycerol ester | partial | 5% | methyl ester | yes |
| 14 | 52% | pentaerythritol | partial | 17% | methyl ester | yes |
| | | ester | | | | |
| 15 | 57% | pentaerythritol | partial | 9% | methyl ester | no |
| | | ester | | | | |

*hydroabietyl alcohol or hydrogenated rosin methyl ester

### EXAMPLES 16-27

### Solid Rosin Esters and Rosin Acid Derivatives in Ethanol

| | Rosin ester | | | Rosin acid | | Stable |
|---|---|---|---|---|---|---|
| Example | Amount | Type | Hydrogenation | Amount | Hydrogenation | solution? |
| 16 | 10% | glycerol | none | 70% | partial | no |
| 17 | 50% | glycerol | none | 25% | partial | yes |
| 18 | 40% | glycerol | none | 30% | partial | yes |
| 19 | 25% | glycerol | partial | 50% | partial | no |
| 20 | 50% | glycerol | partial | 25% | partial | yes |
| 21 | 56% | glycerol | partial | 28% | partial | yes |
| 22 | 50% | glycerol | partial | 25% | high | yes |
| 23 | 50% | glycerol | high | 25% | partial | yes |
| 24 | 56% | glycerol | high | 28% | partial | yes |
| 25 | 50% | glycerol | high | 25% | high | yes |
| 26 | 50% | pentaerythritol | partial | 25% | partial | yes |
| 27 | 50% | pentaerythritol | high | 25% | high | yes |

### EXAMPLES 28-31

### Solid Rosin Esters and Rosin Acid Derivatives in Other Solvents/Solvent Mixtures

| | | Highly | Highly | |
|---|---|---|---|---|
| | | hydrogenated | hy- | |
| | | rosin | drogenated | Stable |
| Example | Solvent(s) | glycerol ester | rosin | solution? |
| 28 | 15% isopropanol | 15% | 70% | no |
| 29 | 12% isopropanol | 66% | 22% | yes |
| 30 | 15% ethanol, | 25% | 50% | yes |
| | 10% isopropanol | | | |
| 31 | 25% ethanol, | 35% | 35% | yes |
| | 5% glycerol | | | |

### EXAMPLE 32

### Solutions with Polymeric Additives

A stable solution was prepared from 35% highly hydrogenated rosin glycerol ester, 15% highly hydrogenated rosin, 15% ethyl cellulose, and 35% ethanol.

### EXAMPLES 33-34

### Solutions of Ethanol in Rosin Esters

| | | Partially | Stable |
|---|---|---|---|
| Example | Ethanol | hydrogenated rosin glycerol ester | solution? |
| 33 | 25% | 75% | yes |
| 34 | 20% | 80% | yes |
| 35 | 2% | 98% | one-phase solid |

### EXAMPLES 36-42

### Viscosity Measurements

Solutions were placed in a glass cylinder (inside diameter=25.4 mm) and equilibrated at 25° C. (±0.2° C.) for 30 min. The time for a soda lime glass ball (diameter=3 mm) to fall between two score lines on the cylinder was recorded. At least seven runs were performed for each sample. From the density of the solution and a calibration curve created from known standards, the viscosity of each sample was calculated.

| Example | Solution | Viscosity | |
|---|---|---|---|
| 36 | 75% natural rosin; 25% ethanol | 300-340 | cP |
| 37 | 50% partially hydrogenated rosin; 25% partially hydrogenated | 350-390 | cP |
| | rosin glycerol ester; 25% ethanol | | |
| 38 | 75% partially hydrogenated rosin glycerol ester; 25% ethanol | 1610-1730 | cP |
| 39 | 49% partially hydrogenated rosin; 24.5% partially hydrogenated | 2120-2400 | cP |
| | rosin glycerol ester; 24.5% ethanol; 2% fumed silica | | |
| 40 | 75% partially hydrogenated rosin glycerol ester; 5% partially | 3780-3940 | cP |
| | hydrogenated rosin methyl ester; 20% ethanol | | |
| 41 | 28% partially hydrogenated rosin; 56% highly hydrogenated rosin | 7790-8150 | cP |
| | glycerol ester; 16% ethanol | | |
| 42 | 88% partially hydrogenated rosin glycerol ester; 12% ethanol | 277,000-333,000 | cP |

### EXAMPLES 43-54

### Film Formation and Adhesion to Human Dentition

Human teeth that had been extracted and stored in a 0.05% chlorhexidine solution for less than two months were rinsed with distilled water, USP. Their roots were embedded in an epoxy support ring, which was allowed to fully cure. The teeth were then conditioned for 1 h in a mechanically stirred, cylindrical, distilled water bath maintained at 37±1° C. The teeth were removed from the water bath and gently patted dry with a cotton towel. Rosin (or other resin) solutions were applied with a nylon-tip brush to the crowns of three teeth per test solution. After the solutions were allowed to dry for a few minutes, the teeth were placed in the stirred water bath for 6 h. The teeth were removed from the water bath and the films were inspected visually and were physically examined with a wooden probe. Before subsequent testing, residual varnish was removed from the teeth with isopropanol. The teeth were dried, conditioned in the water bath, and used as a test substrate as before.

| Example | Varnish |
|---|---|
| 43 | 75% natural rosin; 25% ethanol |
| 44 | 50% partially hydrogenated rosin; 25% partially hydrogenated rosin glycerol ester; 25% |
| | ethanol |
| 45 | 49% partially hydrogenated rosin; 24.5% partially hydrogenated rosin glycerol ester; |
| | 24.5% ethanol; 2% fumed silica |
| 46 | 25% highly hydrogenated rosin; 50% highly hydrogenated rosin glycerol ester; 25% |
| | ethanol |
| 47 | 25% highly hydrogenated rosin; 50% highly hydrogenated rosin pentaerythritol ester; |
| | 25% ethanol |
| 48 | 28% partially hydrogenated rosin; 56% highly hydrogenated rosin glycerol ester; 16% |
| | ethanol |
| 49 | 65% rosin glycerol ester; 10% hydroabietyl alcohol; 25% ethanol |
| 50 | 75% partially hydrogenated rosin glycerol ester; 25% ethanol |
| 51 | 75% partially hydrogenated rosin glycerol ester; 5% partially hydrogenated rosin methyl |
| | ester; 20% ethanol |
| 52 | 35% highly hydrogenated rosin glycerol ester; 15% highly hydrogenated rosin; 15% |
| | ethyl cellulose; 35% ethanol |

Results: All films stayed attached during water treatment and appear smooth and semi-transparent.
Some effort is required to remove thinner films from teeth. Thicker films are softer and less
adherent. Films in Example 49 were slightly softer than the others. Films in Example 50 were semi-
transparent to opaque white and more difficult to remove.

| | |
|---|---|
| Comparative Example 1 | 75% partially hydrogenated rosin glycerol ester; 25% hexanes/ethanol |
| | (70/30 v/v) |

Results: Same as above except that thicker films have rough surface caused by 2 mm diameter
bubbles in film.

| | |
|---|---|
| Comparative Example 2 | 65% refined bleached shellac; 35% ethanol |

Results: Films appear opaque white and textured. A great deal of effort is required to remove the
films from teeth.

### EXAMPLE 53

### Film Formation and Adhesion to Human Gingiva

A solution of 25% ethanol and 75% partially hydrogenated rosin glycerol ester was applied with a cotton swab onto a section of the maxillary gingiva of one of the inventors. The varnish set as a white film and adhered to the gingiva in the moist environment. Gentle rubbing of the film did not cause it to release from the surface. After two hours, the film remained on the gingiva, but was removed with little effort.

### EXAMPLE 54

### Film Formation and Adhesion to Human Epidermis

A solution of 25% ethanol and 75% partially hydrogenated rosin glycerol ester was applied with a cotton swab onto a section of the skin on the back of the hand of one of the inventors. The varnish set as a white film and adhered to the skin through three hand washings. Gentle rubbing of the film did not cause it to release from the surface. After six hours, the film was peeled away by hand.

### EXAMPLE 55

### Fluoride Varnish Preparation and Application

An ethanol solution of highly hydrogenated rosin glycerol ester and partially hydrogenated rosin was combined with a suspension of NaF in ethanol. The NaF suspension was 15 wt. % and had a particle size of 1-7 μm, which was measured on a Horiba CAPA-300 centrifugal particle analyzer. The mixture was heated to reduce the ethanol content. The resulting varnish was a white suspension containing 53% highly hydrogenated rosin glycerol ester, 26% partially hydrogenated rosin, 16% ethanol, and 5% NaF. After two months, some of the NaF had settled. Addition of fumed silica (2% of the total composition) prevented settling, but did not prevent flow when the varnish was spread with an applicator. This varnish also could be dispensed by hand when loaded into a commercial 3-mL plastic syringe fit with a brush tip.

What is claimed is:

1. A varnish composition for topical application to human or animal bodies comprising a solution of a solid rosin ester and at least one natural or modified rosin in a liquid, which is volatile, water soluble, or both, comprising at least one C₂₋₄ alcohol wherein the amount of the solid rosin ester and the natural or modified rosin present in the composition is greater than the solubility of the solid rosin ester alone in the liquid.

2. The composition of claim 1 wherein at least one of the rosins is hydrogenated.

3. The composition of claim 1 wherein the liquid comprises ethanol.

4. The composition of claim 1 wherein the varnish comprises an additional therapeutic agent.

5. The composition of claim 4 wherein the varnish is applied to the oral cavity.

6. The composition of claim 5 wherein the varnish is applied to the dentition.

7. The composition of claim 6 wherein the additional therapeutic agent comprises a fluoride salt.

8. A varnish composition for topical application to human or animal bodies comprising a solution of a solid rosin ester and a liquid, which is volatile, water soluble, or both, comprising at least one C₂₋₄ alcohol, wherein the amount of the solid rosin ester present in the composition is greater than the solubility of the solid rosin ester in the liquid.

9. The composition of claim 8 wherein the solid rosin ester is hydrogenated.

10. The composition of claim 8 wherein the liquid comprises ethanol.

11. The composition of claim 8 wherein the varnish comprises an additional therapeutic agent.

12. The composition of claim 11 wherein the varnish is applied to the oral cavity.

13. The composition of claim 12 wherein the varnish is applied to the dentition.

14. The composition of claim 13 wherein the additional therapeutic agent comprises a fluoride salt.

15. A method of topically applying to human or animal bodies a varnish composition comprising a solution of a solid rosin ester, optionally at least one natural or modified rosin, and a liquid, which is volatile, water soluble, or both, comprising at least one C₂₋₄ alcohol, wherein the amount of the solid rosin ester plus the optional natural or modified rosin present in the composition is greater than the solubility of the solid rosin ester alone in the liquid, directly or indirectly by use of a brush, cloth, gauze, swab, syringe, flexible support tape or patch or matrix, dental tray, or metal, plastic, or wooden applicator.

16. The method of claim 15 wherein at least one of the rosins is hydrogenated.

17. The method of claim 15 wherein the liquid comprises ethanol.

18. The method of claim 15 wherein the varnish comprises an additional therapeutic agent.

19. The method of claim 18 wherein the varnish is applied using a syringe.

20. The method of claim 18 wherein the varnish is applied using a flexible support that is optionally removed after varnish application.
